(19) United States
(12) Patent Application Publication
(10) Pub. No.: US 2015/0125552 A1
(43) Pub. Date: May 7, 2015

(54) NO/Nitrogen Gaseous Mixture With A High NO Content For The Treatment Of Severe Hypoxemic Respiratory Failure

(71) Applicant: AIR LIQUIDEE SANTE (INTERNATIONAL), Paris (FR)

(72) Inventors: Pierre De Villemeur, Louveciennes (FR); Laurent Lecourt, Cachan (FR)

(21) Appl. No.: 14/400,562

(22) PCT Filed: Apr. 15, 2013
(86) PCT No.: PCT/FR2013/050817

§ 371 Date: Nov. 12, 2014

(30) Foreign Application Priority Data
May 24, 2012 (FR) 1254765

(51) Int. Cl. A61K 33/00; A61K 9/00

(52) U.S. Cl. CPC A61K 33/00; A61K 9/007
USPC 424/718

(57) ABSTRACT

The invention concerns a gaseous medicine comprising a mixture of nitrogen monoxide (NO) and nitrogen (N2) for use by inhalation for the treatment of hypoxemic respiratory failure linked to pulmonary vasoconstriction in humans, said gaseous mixture containing between 1200 and 4500 ppm by volume (ppmv) of nitrogen monoxide (NO) and said hypoxemic respiratory failure being chosen from acute respiratory distress syndrome (ARDS) or persistent pulmonary hypertension of the newborn infant (PPHN).

### CROSS REFERENCE TO RELATED APPLICATIONS

This application is a 371 of International Application PCT/FR2013/050817 filed Apr. 15, 2013, which claims priority to French Application No. 1254765 filed May 24, 2012, the entire contents of which are incorporated herein by reference.

### BACKGROUND

The invention relates to a gaseous medicament based on nitrogen monoxide (NO) and between 1200 and 4500 ppm by volume of nitrogen monoxide (NO), used by inhalation for treating hypoxemic respiratory distress linked to pulmonary vasoconstriction in humans, such as ARDS or persistent pulmonary hypertension of the newborn (PPHN).

Hypoxemia is the medical term used for describing the decrease in the amount of oxygen contained in the blood. Hypoxemia may be the result of certain respiratory pathologies, such as acute respiratory distress syndrome (ARDS) or primary pulmonary hypertension of the newborn (PPHN).

ARDS is a critical condition that is life-threatening for the person who is suffering therefrom, in which the pulmonary compliance and the capacity for gas exchanges drop radically, which results in a deterioration of the alveolar-capillary membrane leading to a pulmonary edema.

The most commonly used definition of ARDS is the one from the following document: The American-European Consensus Conference on ARDS, Am J Respir Crit Care Med. 1994 March; 149 (3 Pt 1): 818-24. According to this document, ARDS is characterized by:
a clinical condition of the patient with respiratory distress of acute onset,
disturbed blood gases with PaO₂/FiO₂ ratio<200,
bilateral infiltrate on lung radiograph,
non-cardiogenic pulmonary edema (PAOP≦18 mm Hg), and
good echocardiographic function or no clinical signs of heart failure.

Furthermore, PPHN is a form of pulmonary hypertension observed in newborns, the cause of which is a persistence of pulmonary artery vasospasm after birth. In fact, during pregnancy, the lungs of the fetus are not used for gas exchanges, i.e. intake of oxygen and removal of CO₂, and fetal circulation sends most of the blood far from the lungs owing to special connections in the heart and the large blood vessels.

### SUMMARY

At birth, the newborn begins to breathe air. The change in pressure in the lungs that results therefrom then normally helps to make good the connections of the newborn and to redirect the blood flow, that is to say that the blood is pumped to the lungs in order to help with the gas exchanges. However, when the newborn has a low level of oxygen or respiratory difficulties at birth, these changes may not occur and the blood circulation continues to take place as before the birth, that is to say that the blood is directed far from the lungs but, in this case, the pressure of the lungs remains high.

If the blood is sent far from the lungs, the latter cannot carry out the gas exchanges, that is to say the capture of oxygen and the removal of carbon dioxide, even if the newborn is supplied with 100% by volume of oxygen.

Babies suffering from PPHN therefore have low blood oxygen levels, which may be dramatic insofar as all the organs of the body depend on oxygen-rich blood and, hence, in the case of insufficient blood perfusion, they may be damaged by lack of oxygen.

In both cases, the patient suffers hypoxemic respiratory distress linked to a pulmonary vasoconstriction, that is to say a vasoconstriction of the blood vessels of the lungs leading to impaired pulmonary gas exchanges and a poor blood supply of the lungs.

Currently, one treatment known for treating pathologies of this type consists of an administration, by inhalation, of NO gas to the patient.

Thus, Kinox™ is a gaseous medicament formed from a mixture of NO (225 or 450 ppm by volume) and nitrogen indicated, in combination with assisted ventilation and conventional treatment, in the treatment of newborns having a gestational age of at least 34 weeks exhibiting hypoxemic respiratory distress combined with clinical or echocardiographic signs of pulmonary arterial hypertension, with the purpose of improving oxygenation and avoiding recourse to extracorporeal oxygenation. The dosage is determined by the clinical condition of the patient but the maximum recommended dose is 20 ppm by volume.

Furthermore, documents EP-560928, EP-A-1516639 and EP-A-786264 recommend using NO/N₂ gas mixtures that contain up to 800 ppm by volume of NO.

Just before it is administered, the NO/N₂ mixture is diluted with a gas containing at least 21% oxygen, such as air, O₂-enriched air or an N₂/O₂ mixture, so as to make the mixture thus obtained non-hypoxic. This dilution makes it possible to decrease the concentration of NO in the gas administered to less than 40 ppmv, i.e. to the dosage desired for the patient in question. This is generally carried out in the patient circuit of a ventilator.

However, in order to minimize the formation of toxic NO₂ by reaction of NO with oxygen, the mixture obtained containing NO and oxygen cannot be stored for long time and must be administered immediately after having been mixed. This is conventionally carried out in the ventilation circuit of a ventilation device, that is to say a respirator.

It follows therefrom that with the known NO/N₂ mixtures containing 225, 450 or 800 ppmv, mixing with air or an oxygen-rich gas leads not only to a dilution of the NO but also inevitably of the oxygen which will lead to a reduction in the concentration of O₂ in the mixture administered to the patient.

By way of example, an NO/N₂ mixture containing 800 ppmv of NO mixed with air (O₂ content of 21% by vol.) that must be administered by inhalation to a patient at a final concentration of 40 ppmv of NO, will have to undergo a 20-times dilution, namely 1 volume of NO/N₂ per 19 volumes of air. The air will then also undergo a dilution (dilution factor of 19/20) and the final O₂ content will be less than 20%, therefore less than the content in the ambient air.

Yet patients suffering from hypoxemic respiratory distress already have an insufficient blood oxygen content.

Hence, it is understood that the dilution of the oxygen with the NO/N₂ mixture may be problematic for the patient.

In view of this, the problem that is addressed is to propose an inhalable gaseous medicament based on NO for which the dilution effect will be lower than with the existing mixtures.

In other words, the present invention aims not to lower the FiO₂ provided to the patient due to a great dilution of the oxygen with the gas containing the NO, while guaranteeing an effective treatment of the vasoconstrictions linked to hypoxemiac respiratory distresses of PPHN, ARDS or other type.

The solution of the invention is then a gaseous medicament comprising a mixture of nitrogen monoxide (NO) and nitrogen (N₂) for use by inhalation for treating hypoxemic respiratory distress linked to a pulmonary vasoconstriction in humans, said gas mixture containing between 1200 and 4500 ppm by volume (ppmv) of nitrogen monoxide (NO) and said hypoxemic respiratory distress being selected from acute respiratory distress syndrome (ARDS) or persistent pulmonary hypertension of the newborn (PPHN).

Depending on the case, the medicament of the invention may comprise one or more of the following technical features:
the NO/N₂ mixture is mixed, before the inhalation thereof, with an additional gas containing at least 21% by volume of oxygen.
the additional gas contains at least 25% by volume of oxygen.
the additional gas contains at least 30% by volume of oxygen.
the additional gas contains at least 40% by volume of oxygen.
the additional gas contains at least 50% by volume of oxygen.
the additional gas contains at least 60% by volume of oxygen.
the additional gas contains at least 70% by volume of oxygen.
the additional gas contains at least 80% by volume of oxygen.
the additional gas contains from 90% to 100% by volume of oxygen.
the additional gas is selected from oxygen, air, air/oxygen mixtures, that is to say air to which oxygen or an oxygen-containing gas has been added, or nitrogen/oxygen mixtures.
the NO/N₂ gas mixture contains at least 1300 ppmv of NO, preferably at least 1400 ppmv of NO, and nitrogen (N₂) for the remainder.
the NO/N₂ gas mixture contains at least 1500 ppmv of NO and nitrogen (N₂) for the remainder.
the NO/N₂ gas mixture contains less than 3500 ppmv of NO.
the NO/N₂ gas mixture contains between 1500 and 4500 ppmv of NO.
the NO/N₂ gas mixture contains from 1500 to 2500 ppmv of NO.
the NO/N₂ gas mixture is contained in a container having an internal volume of less than or equal to 12 liters (water equivalent).
the container containing the NO/N₂ gas mixture is of cylindrical shape and comprises, at one end, a base and, at the other end, a neck with an outlet orifice, attached to which is a device for controlling the gas flow and/or for reducing the pressure.
the NO/N₂ gas mixture is contained in a gas cylinder.
the container containing the NO/N₂ gas mixture comprises a neck with an outlet orifice, attached to which is an integrated valve regulator.
the NO/N₂ gas mixture is stored in a container at a pressure of from 100 to 500 bar absolute, preferably between 140 and 350 bar absolute.
the NO/N₂ gas mixture is stored in a container equipped with a valve or an integrated valve regulator.
the NO/N₂ gas mixture is stored in a container of cylindrical shape having a diameter between 5 and 40 cm and a height between 10 and 80 cm.
the human treated is an adult, child or a newborn.
the NO/N₂ gas mixture is diluted with an oxygen-containing gas in a ventilation circuit of a respirator or a medical ventilator.
the NO/N₂ gas mixture is delivered continuously into the ventilation circuit of the respirator.
alternatively, the NO/N₂ gas mixture is delivered in a pulsed manner into the ventilation circuit of the respirator.

### BRIEF DESCRIPTION OF THE DRAWING

The present invention will now be better understood owing to the description given below with reference to the appended FIGURE.

FIG. 1 represents one embodiment of an installation for dispensing NO that can be used within the context of the present invention.

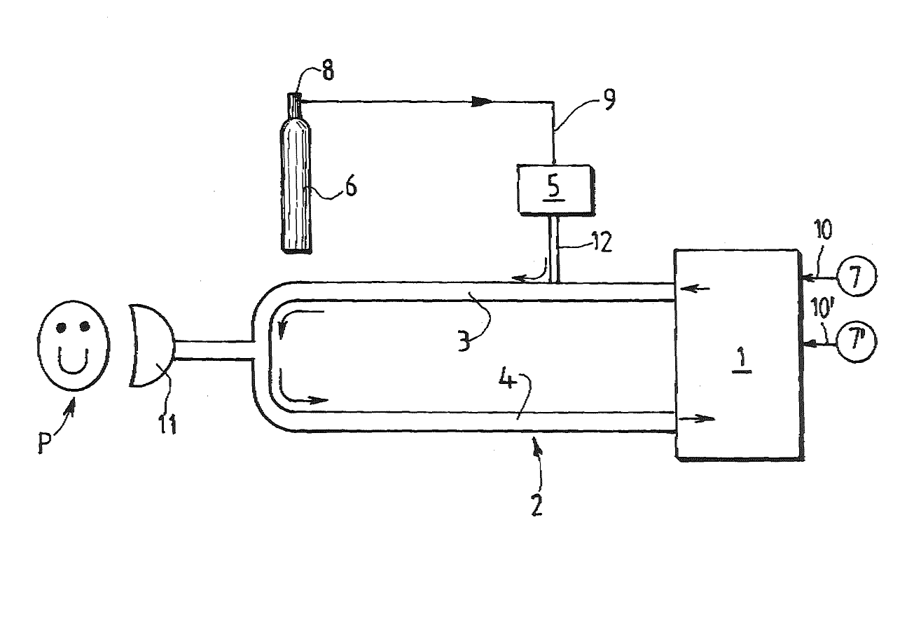

### DESCRIPTION OF PREFERRED EMBODIMENTS

Seen therein is an installation for dispensing NO that can be used to administer a gaseous mixture of NO and nitrogen at high concentration, that is to say 1200 ppm or more, preferably between 1500 and 4500 ppm, to a patient P with hypoxemic respiratory distress with an FiO₂ of around 100%, for example an adult suffering from ARDS or a newborn suffering from persistent pulmonary hypertension.

This installation comprises a ventilator 1 comprising a respiratory circuit or patient circuit 2 with two branches, that is to say with an inhalation branch 3 and an exhalation branch 4. The inhalation branch 3 is designed to convey respiratory gas from the ventilator 1 to the patient P, whereas the exhalation branch 4 is designed to convey the gas exhaled by the patient P to the ventilator 1.

At the patient P, the gas is administered by means of a patient interface 11, for example a respiratory mask or a tracheal cannula or catheter.

The ventilator 1 is supplied, via a first delivery line 10, with air (O₂ content of 21% by volume) originating from an air source 7 and via a second delivery line 10′ with oxygen resulting from an oxygen source 7′, such as an oxygen cylinder or a duct transporting oxygen originating from an oxygen production unit, such as a pressure swing (PSA) unit, or from an oxygen storage unit, such as a buffer or storage tank.

The oxygen-rich gas is delivered by the ventilator 1 into the inhalation branch 3 of the patient circuit 2.

Furthermore, a device 5 for dispensing NO is fluidically connected to said inhalation branch 3 of the patient circuit 2 in order to deliver therein, via a feed line 12, an NO/N₂ mixture containing a high concentration of NO, that is to say at least 1200 ppm by volume, according to the present invention.

The device 5 for dispensing NO is itself supplied with an NO/N₂ mixture, via a gas feed line 9, by a source of NO 6 such as a gas cylinder equipped with a valve or integrated valve regulator 8, preferably protected by a cap for protection against impacts.

The device 5 for dispensing NO makes it possible in particular to control the amount of NO/N₂ released into the inhalation branch 3, and also the method of releasing this mixture, that is to say continuously or in a pulsed manner, for example only during the inhalation phases of the patient P.

Therefore, in the inhalation branch 3, a dilution of the NO/N₂ mixture with the oxygen-rich gas dispensed by the ventilator 1 takes place.

The dilution depends on the content of the initial NO/N₂ mixture but also on the concentration of gas to be administered to the patient.

The NO/N₂ gas mixture according to the invention is therefore an inhalable medicament that can be used in combination with an assisted ventilation 1 for the treatment in particular of newborns having a gestational age of at least 34 weeks exhibiting hypoxemic respiratory distress combined with clinical or echocardiographic signs of pulmonary arterial hypertension, with the purpose of improving oxygenation and avoiding recourse to extracorporeal oxygenation.

It may also be used for treating adults or children suffering from ARDS.

The examples below give examples of mixtures that can be used in patients suffering from ARDS or PPHN.

### Example 1

### Adult Suffering from ARDS

| | |
|---|---|
| | Comparative |
| Invention | (prior art) |

| | | | | |
|---|---|---|---|---|
| NO content of the initial | 2000 | ppmv | 450 | ppmv |
| NO/N₂ mixture | | | | |
| Ventilation | 10 | l/min | 10 | l/min |

| | | |
|---|---|---|
| Ventilator set point FiO₂ | 60% | 60% |

| | | | | |
|---|---|---|---|---|
| Final content to be | 20 | ppmv | 20 | ppmv |
| administered to the patient | | | | |

| | | |
|---|---|---|
| Dilution factor | 1% | 4.5% |
| Actual FiO₂ after dilution | 59% | 55.4% |

### Example 2

### Newborn Suffering from PPHN

| | | | Comparative | | Comparative | |
|---|---|---|---|---|---|---|
| | Invention | | (prior art) | | (prior art) | |
| NO content of the initial | 2000 | ppmv | 450 | ppmv | 225 | ppmv |
| NO/N₂ mixture | | | | | | |
| Ventilation | 2 | l/min | 2 | l/min | 2 | l/min |
| Ventilator set point FiO₂ | 100% | | 100% | | 100% | |
| Final content to be | 20 | ppmv | 20 | ppmv | 20 | ppmv |
| administered to the patient | | | | | | |

| | | | |
|---|---|---|---|
| Dilution factor | 1% | 4.5% | 9.08% |
| Actual FiO₂ after dilution | 98.2% | 92.3% | 85% |

Seen in tables 1 and 2 is the advantage of using high concentrations of NO in the initial mixture, all the more so when the target dosages of NO are high (e.g. non-responsive patient requiring dosages of greater than 20 ppmv) and when the required FiO₂ values are also high, that is to say of the order of 90% to 100%.

Indeed, such high FiO₂ values cannot be maintained for excessively low initial concentrations, that is to say of less than 1000 ppmv approximately, and in this case, the positive effects of the NO are offset by the deleterious effects of the dilution on the FiO₂.

Generally, the invention also relates to a therapeutic treatment method, wherein a gaseous medicament comprising a mixture of nitrogen monoxide (NO) and nitrogen (N₂) is administered by inhalation to a patient suffering from hypoxemic respiratory distress linked to pulmonary vasoconstriction, such as ARDS, or PPHN, said gas mixture containing at least 1200 ppm by volume (ppmv) of nitrogen monoxide (NO), and said patient being an adult, a child or a newborn.

Prior to being administered to the patient, the mixture of nitrogen monoxide (NO) and nitrogen (N₂) is diluted with an oxygen-containing gas, preferably this dilution is carried out in the ventilation circuit of a medical ventilator or the like.

Generally, table 1 below shows the volumes (in ml) of NO/N₂ administered in the inhalation branch 3 of the patient circuit 2 of the ventilator 1 for a volume per minute of 10 l/min of the ventilator (i.e. air/O₂), in order to obtain NO concentrations between 5 and 40 ppmv, as a function of the NO concentration in the cylinder containing the NO/N₂ mixture, i.e. ranging from 100 to 3500 ppmv.

TABLE 1

| | NO concentration in the cylinder (ppmv) | | | | | | | | | | | |
|---|---|---|---|---|---|---|---|---|---|---|---|---|
| NO final dose | 100 | 200 | 225 | 400 | 450 | 800 | 1000 | 1500 | 2000 | 2250 | 2700 | 3500 |
| 5 ppmv | 556 | 256 | 227 | 127 | 112 | 63 | 50 | 33 | 25 | 22 | 19 | 14 |
| 10 ppmv | 1111 | 513 | 455 | 253 | 225 | 126 | 101 | 67 | 50 | 45 | 37 | 29 |
| 20 ppmv | 2222 | 1026 | 909 | 506 | 449 | 252 | 201 | 134 | 100 | 89 | 74 | 57 |
| 40 ppmv | 4444 | 2051 | 1818 | 1013 | 899 | 503 | 402 | 268 | 201 | 178 | 148 | 114 |

As can be seen in table 1, the volumes of NO administered in order to obtain doses of 5 ppmv are respectively 556 ml for a concentration of 100 ppmv of NO and 14 ml for a concentration of 3500 ppmv. Likewise, for doses of 40 ppmv, the volumes are 4444 ml for a concentration of 100 ppmv of NO and 114 ml for an NO concentration of 3500 ppmv.

This clearly demonstrates the advantage that there is in using high NO concentrations, that is to say of at least 1200 ppmv, in order to reduce the impact of the volume of NO administered and of the associated dilution on the ventilator parameters (see table 2) since the higher the concentration, the lower the volume of NO required.

Furthermore, table 2 illustrates the dilution percentages (%) obtained for NO concentrations from 100 to 3500 ppmv in order to obtain doses ranging from 5 to 40 ppmv, under the same administration conditions as those from table 1 (i.e. ventilation of 10 l/min).

TABLE 2

| | NO concentration in the cylinder (ppmv) | | | | | |
|---|---|---|---|---|---|---|
| NO final dose | 100 | 200 | 225 | 400 | 450 | 800 |
| 5 ppmv | 5.56% | 2.56% | 2.27% | 1.27% | 1.12% | 0.63% |
| 10 ppmv | 11.11% | 5.13% | 4.55% | 2.53% | 2.25% | 1.26% |
| 20 ppmv | 22.22% | 10.26% | 9.09% | 5.06% | 4.49% | 2.52% |
| 40 ppmv | 44.44% | 20.51% | 18.18% | 10.13% | 8.99% | 5.03% |

| 1000 | 1500 | 2000 | 2250 | 2700 | 3500 |
|---|---|---|---|---|---|
| 0.50% | 0.33% | 0.25% | 0.22% | 0.19% | 0.14% |
| 1.01% | 0.67% | 0.50% | 0.45% | 0.37% | 0.29% |
| 2.01% | 1.34% | 1.00% | 0.89% | 0.74% | 0.57% |
| 4.02% | 2.68% | 2.01% | 1.78% | 1.48% | 1.14% |

As can be seen, for a same treatment set point, the dilution becomes negligible for high NO concentrations, that is to say of at least 1200 ppmv, and hence no longer influences the ventilator parameters and set points. Indeed, the lower the volume of NO, the less it will change the ventilation set points and counteract the beneficial effects of the treatment, in particular the FiO₂.

According to the invention, the use of a gas mixture containing at least 1200 ppm by volume (ppmv) of nitrogen monoxide (NO), typically containing from 1200 to 4500 ppmv, is therefore recommended.

Lastly, tables 3 to 5 below show the impact of the dilutions on the desired FiO₂ values for initial NO concentrations in the cylinder of 225, 450 and 2000 ppmv.

The FiO₂ is a very important parameter within the context of the illnesses treated by NO (neonatal refractory hypoxemia, ARDS, etc.) given that for these illnesses FiO₂ values of the order of 100% may be necessary.

This implies that the dilution must be as low as possible in order to retain an FiO₂ value as high as possible and therefore the beneficial effect of the treatment by inhaled NO.

TABLE 3

| | FiO₂ desired (%) | | | | |
|---|---|---|---|---|---|
| 225 ppmv NO | 21 | 23 | 40 | 60 | 100 |
| | FiO₂ obtained (%) | | | | |
| 5 ppmv | 20.52 | 22.48 | 39.09 | 60.00 | 97.73 |
| 10 ppmv | 19.59 | 21.46 | 37.31 | 57.27 | 93.29 |
| 20 ppmv | 17.81 | 19.51 | 33.92 | 52.07 | 84.80 |
| 40 ppmv | 14.57 | 15.96 | 27.75 | 42.60 | 69.39 |

TABLE 4

| | FiO₂ desired (%) | | | | |
|---|---|---|---|---|---|
| 450 ppmv NO | 21 | 23 | 40 | 60 | 100 |
| | FiO₂ obtained (%) | | | | |
| 5 ppmv | 20.76 | 22.74 | 39.55 | 59.33 | 98.88 |
| 10 ppmv | 20.30 | 22.23 | 38.66 | 57.99 | 96.65 |
| 20 ppmv | 19.39 | 21.23 | 36.92 | 55.39 | 92.31 |
| 40 ppmv | 17.64 | 19.32 | 33.61 | 50.41 | 84.01 |

TABLE 5

| | FiO₂ desired (%) | | | | |
|---|---|---|---|---|---|
| 2000 ppmv NO | 21 | 23 | 40 | 60 | 100 |
| | FiO₂ obtained (in %) | | | | |
| 5 ppmv | 20.95 | 22.94 | 39.90 | 59.85 | 99.749 |
| 10 ppmv | 20.84 | 22.83 | 39.70 | 59.55 | 99.249 |
| 20 ppmv | 20.63 | 22.60 | 39.30 | 58.95 | 98.254 |
| 40 ppmv | 20.22 | 22.15 | 38.51 | 57.77 | 96.284 |

As can be seen in tables 3 and 4 relating to low initial contents of NO, namely 225 and 450 ppmv of NO (remainder nitrogen), the decreases of FiO₂ caused by the dilution may represent around 20% to 30% of the desired FiO₂ (i.e. the set point FiO₂), which becomes prejudicial to the treatment by NO since a low FiO₂ value cancels out the positive effects of the NO.

Conversely, as shown in table 5, using a high concentration of NO (here 2000 ppm of NO in nitrogen) does not modify or else negligibly modifies (i.e. around <1%) the FiO₂ set points thus making it possible to treat patients with O₂ contents of almost 100% over short periods, in order to get through certain extreme situations.

Although an increase in the concentration of NO in the cylinders may appear simple at first glance, it does entail problems linked to the potential toxicity of high contents of NO (>800 ppmv), since any excessive dose, due to a poor dilution for example, may result in a toxic dose being administered to the patient and since, furthermore, the higher the concentration of NO, the greater the risk of forming toxic compounds, such as toxic NO₂ that is formed by oxidation of NO in the presence of oxygen.

In other words, the solution proposed by the invention goes against certain prejudices that exist in the field in question, which consider that the use of NO at high doses, that is to say greater than 1200 ppmv, is dangerous and not recommended.

It will be understood that many additional changes in the details, materials, steps and arrangement of parts, which have been described herein in order to explain the nature of the invention, may be made by those skilled in the art within the principle and scope of the invention as expressed in the appended claims. Thus, the present invention is not intended to be limited to the specific embodiments in the examples given above.

1-14. (canceled)

15. A method of treating a hypoxemic respiratory distress linked to a pulmonary vasoconstriction in humans and selected from acute respiratory distress syndrome (ARDS) or persistent pulmonary hypertension of the newborn (PPHN), comprising the step of administering by inhalation a gas mixture containing between 1200 and 4500 ppm by volume (ppmv) of nitrogen monoxide (NO) to said human, said human suffering from the hypoxemic respiratory distress linked to a pulmonary vasoconstriction in humans and selected from acute respiratory distress syndrome (ARDS) or persistent pulmonary hypertension of the newborn (PPHN).

16. The method of claim 15, wherein the NO/N₂ gas mixture contains 1300 ppmv to 4500 ppmv of NO, and nitrogen (N₂) for the remainder.

17. The method of claim 15, wherein the NO/N₂ gas mixture contains from 1200 ppmv to 3500 ppmv of NO.

18. The method of claim 15, wherein the NO/N₂ mixture is mixed, before the inhalation thereof, with an additional gas containing at least 21% by volume of oxygen.

19. The method of claim 18, wherein the additional gas contains at least 50% by volume of oxygen.

20. The method of claim 18, wherein the additional gas contains from 90% to 100% by volume of oxygen.

21. The method of claim 18, wherein the additional gas is selected from oxygen, air, air/oxygen mixtures and nitrogen/oxygen mixtures.

22. The method of claim 16, wherein the NO/N₂ gas mixture contains at least 1400 ppmv to 4500 ppmv of NO, and nitrogen (N₂) for the remainder.

23. The method of claim 16, wherein the NO/N₂ gas mixture contains from 1500 to 2500 ppmv of NO.

24. The method of claim 16, wherein the NO/N₂ gas mixture is contained prior to administration by inhalation in a container having an internal volume of less than or equal to 12 liters (water equivalent).

25. The method of claim 16, wherein the NO/N₂ gas mixture is stored in a container at a pressure of from 100 to 500 bar.

26. The method of claim 24, wherein the NO/N₂ gas mixture is stored in a container equipped with a valve or an integrated valve regulator.

27. The method of claim 24, wherein the NO/N₂ gas mixture is stored in a container of cylindrical shape having a diameter between 5 and 40 cm and a height between 10 and 80 cm.

28. The method of claim 15, wherein the human treated is an adult, a child or a newborn.
